(19) United States
(12) Patent Application Publication
(10) Pub. No.: US 2024/0165316 A1
(43) Pub. Date: May 23, 2024

(54) SMOKE EVACUATION MODULE AND A METHOD OF CALIBRATING THE SMOKE EVACUATION MODULE

(71) Applicant: CFN MEDICO APS, Valby (DK)

(72) Inventor: Christian Ollendorff, Kolding (DK)

(21) Appl. No.: 18/283,956

(22) PCT Filed: Mar. 25, 2022
(86) PCT No.: PCT/DK2022/050061

§ 371 Date: Sep. 25, 2023

(30) Foreign Application Priority Data
Mar. 26, 2021 (DK) PA 2021 70149

(51) Int. Cl. A61M 1/00; A61B 17/32

(52) U.S. Cl. CPC A61M 1/80; A61B 17/32

(57) ABSTRACT

A smoke evacuation module and a calibration method thereof is provided, where the smoke evacuation module is adapted to be installed in a supply column using a plug-and-play configuration. The control system has a controller and a socket interface for connecting one or more surgical tools. The pump system has a pump and an inlet interface which is further connected to the one or more surgical tools. The control system is capable of controlling a suction for each surgical tool connected to the smoke evacuation module, thus allowing more than one surgical tool to be used at the same time.

### CROSS-REFERENCE TO RELATED APPLICATIONS

This application claims priority to PCT Application No. PCT/DK2022/050061, having a filing date of Mar. 25, 2022, which is based on DK Application No. PA 2021 70149, having a filing date of Mar. 26, 2021, the entire contents both of which are hereby incorporated by reference.

### FIELD OF TECHNOLOGY

The following relates to a smoke evacuation module comprising a pump system connected to an inlet interface and an outlet interface, wherein the pump system is configured to generate a fluid flow from the inlet to the outlet. The inlet interface is configured to operatively engage with a surgical tool via a fluid conduit. The operation of the smoke evacuation module is controlled by a control system, which is configured to operate in multiple suction modes.

### BACKGROUND

It is known to use smoke evacuation systems to remove surgical smoke from an operation site, more specifically when using electrosurgery. This is particularly desired during open surgery as the surgical smoke is carcinogenic and has a foul smell. There is also a desire to remove surgical smoke during closed surgery as the smoke will accumulate inside the cavity and obscure the video image presented to the surgent.

U.S. Pat. No. 6,592,543 B1 discloses a smoke evacuation system for passively evacuating smoke during endoscopic surgery using a filter system and a flow regulator.

U.S. Pat. No. 5,578,000 A discloses a smoke evacuation system for use during endoscopic surgery, comprising a fluid conduit system coupled to an outlet on the trocar at one end and to an inlet on a wall vacuum system or a stand-alone smoke evacuation unit.

Modern operation rooms may comprise a central suction apparatus, from which flexible suction hoses and power cables extend to the operation site. Cleaning and installation of such apparatus is complex and time consuming. The apparatus also requires ceiling mounted sensors and a signal box mounted on a trolley or ceiling mounted guide rail, which present an obstacle for nurses and surgeons. Also, the apparatus requires add-on devices for use during laparoscopy. Such apparatus is also sensitive to breakdowns as operations need to be cancelled during repair or maintenance.

The Surgical smoke plume evacuator form Skytron is shaped as a cassette intended for mounting on a shelf on a ceiling mounted equipment carrier. The smoke evacuator comprises a removable filtering unit which must be replaced after 35 hours operation. The smoke evacuator can be switched between three suction levels and the filtering unit has three separate inlets for connection of a fluid conduit. The smoke evacuator may be used for either open surgery or laparoscopy and is activated by mounting an automatic activation device to the smoke evacuator. The automatic activation device is a current clamp measuring the power consumption of the electrical wire connected to the cutting tool. Such current clamps cannot activate all types of cutting tools and cannot be used for laser surgery.

Alternatively, the various equipment for open or closed surgery may be arranged on an equipment cart. The equipment cart may be fitted with slidable cassettes or shelves on the consoles or modules are placed. An example of such a console is disclosed in U.S. Pat. No. 5,417,246 A which discloses a pressure control system for controlling the gas pressure provided to the surgical instruments which is activated by a foot pedal. Another example is disclosed in WO 2019/186502 A1 which discloses a smoke evacuation cassette comprising a compressor for providing suction to the inlet, wherein a robotic unit may be coupled to the equipment cart. The controller inside the smoke evacuation cassette can switch between different operation modes.

WO 2019/130117 A1 discloses an equipment cart fitted with a number of slidable cassette shaped modules, each comprising a docking console for engaging a matching docking console in the equipment cart. One cassette may be a smoke evacuation module fitted with a single inlet adapted for open surgery, an onboard motor and a carbon reservoir. Onboard sensors detect the quality of the smoke and the onboard processor regulate the suction accordingly.

WO 97/14364 A1 discloses a smoke evacuation system comprising an electrosurgical device coupled to a standalone blower or a wall suction source, wherein radio frequency based current sensors are used to measure the current in the electrosurgical device. This smoke evacuation system cannot be used for laparoscopy and the current sensors cannot communicate with laser or bipolar equipment.

Therefore, there is a need for an improved smoke evacuator that is easy to install and be operated with different types of surgical tools.

### SUMMARY

An aspect relates to a smoke evacuation module that solves the abovementioned problems of the conventional art.

An aspect of embodiments of the present invention is to provide a smoke evacuation module that is quick and easy to install and is capable of functioning with known types of supply columns.

An aspect of embodiments of the present invention is to provide a smoke evacuation module that has an increased versatility and requires a minimum of servicing.

An aspect of embodiments of the present invention is to provide a smoke evacuation module that can be recalibrated to function with different types of surgical tools.

An aspect of embodiments of the present invention is achieved by a smoke evacuation module, which is adapted for plug-and-play engagement with a supply column and comprising:
a pump system connected to an inlet interface at one end and to an outlet interface at the other end, the pump system comprising a pump configured to generate a suction flow from the inlet interface to the outlet interface,
wherein the inlet interface is configured to operatively engage with at least one surgical tool,
wherein the outlet interface is configured to engage with an exhaust; and
a control system configured to be connected to the pump system, the control system comprising a controller configured to operate the pump in at least two different suction modes.

This provides a fast and easy installation of the smoke evacuation module compared to conventional smoke evacuation modules. Embodiments of the present invention can be installed in 1-2 hours by a technician. Embodiments of the present invention also allow for fewer smoke evacuation modules at the operation rooms. Embodiments of the present invention also provide more space for nurses and surgeons. No active filtration systems are needed, this saves costs and maintenance.

According to an embodiment, the smoke evacuation module is configured to be installed inside the supply column, wherein at least the pump system or the control system is configured to be coupled to the supply column.

This provides a compact solution as the smoke evacuation module is intended to be integrated into the supply column. Thereby reduces the need for an equipment cart as the pump and control systems are arranged in the supply column. The pump system and/or the control system are simply positioned in at least one cavity in the supply column and coupled to the air source, the main power lines, and/or the exhaust system located in the supply column. Optionally, in some embodiments, one or more front panels are used to close off the cavities so the smoke evacuation module is concealed inside the supply column. This allows for a fast and easy installation of the smoke evacuation module, suitable for installation in supply columns from different manufactures.

According to an embodiment, the pump is at least one vacuum ejector.

Foul smells and toxic particles may thereby be removed from the area around the patient using the vacuum ejector. This may also prevent the surgical smoke from obscuring the endoscopic camera. The use of a vacuum ejector allows for an instant start of the suction compared to conventional pumps and motors; thus, the smoke evacuation module can be used at once as no ramp-up time is needed.

A single vacuum ejector may be used to generate suctions for each inlet. A vacuum regulator may then be arranged at each inlet to individually regulate the suction of each inlet. Alternatively, a separate vacuum ejector may be used for each inlet and the vacuum regulators may be omitted.

This allows for individual control of each suction of the smoke evacuation module.

According to an embodiment, the pump is powered by an external air source configured to supply compressed air to the pump.

Embodiments of the present invention may have a very low noise level, thereby providing a pleasant working environment for the people in the operation room. Conventional smoke evacuation modules use an electrical motor generating a high noise level, thus causing a discomfort to the people in the operating room. The use of an external air source allows for a compact design as no integrated compressor or motor is needed. The present smoke evacuation module may be operated at a reduced pressure of the compressed gas (e.g., air), for example, at least 3 bars, e.g., between 4-5 bars.

According to an embodiment, the controller is configured to perform a calibration procedure, wherein the smoke evacuation module is calibrated to function correctly with the at least one surgical tool in either of the at least two different suction modes.

Embodiments of the present invention allow for a fast and simple calibration or recalibration of the smoke evacuation module compared with conventional smoke evacuation modules. The controller may determine a standby parameter of the surgical tool connected to a selected inlet/socket, thus no need for transmitting data packages between the surgical tool and the controller. The present smoke evacuation module may thereby be calibrated to operate correctly together with a particular surgical tool, thus reducing the risk of errors during operation. The present smoke evacuation module may function with any electrosurgical tools from different manufactures. Conventional smoke evacuation modules are typically limited to function only with surgical tools from the same manufacture, where a handshake or authorisation process is performed to calibrate the surgical tool.

According to an embodiment, the inlet interface comprises at least two inlets each adapted to be coupled a dedicated surgical tool, for example, in embodiments, the surgical tool may be selected from a group of lasers, electrocautery devices, electrocautery devices, diathermy devices or robotic devices.

Embodiments of the present invention allow multiple surgical tools to connect to the smoke evacuation module. Each inlet may be adapted to fit electrosurgical tools from different manufactures. Any type of surgical tool may be coupled to the smoke evacuation module in which a suction effect is desired. The surgical tool may be a laser, an electrocautery device, an electrocautery device, a diathermy device, a robotic device, a suction device or any other device used in a surgical procedure.

Alternatively, a suction tube, e.g., fitted with a suction guard or tip for preventing aspiration of objects, may be coupled to the smoke evacuation module. This increases the versatility of the present smoke evacuation module.

According to an embodiment, the pump is configured to operatively engage at least two surgical tools coupled to the inlet interface at the same time, where the controller is configured to independently control the respective suctions provided to the at least two surgical tools.

A single surgical tool may be connected and operated according to a selected suction mode. Further, the present smoke evacuation module may be capable of operating multiple surgical tools at the same time, either simultaneously or independently. The multiple surgical tools may be of the same type or of different types, thus surgical tools of different manufactures or different surgical tools may be used at the same time.

According to an embodiment, the pump system comprises a first valve element connected to the pump and further to a first inlet at the inlet interface, wherein the controller is configured to control the first valve element to provide a first suction adapted to closed surgery.

A first suction mode may be adapted for minimally-invasive surgical procedures, such as endoscopic surgery. The term “endoscopic” should here be understood as arthroscopic, laparoscopic, hysteroscopic, thoracoscopic or any other closed surgical procedure performed with tools inserted through small incisions the patient.

A flexible fluid conduit, e.g., a suction tube, may be connected to a first inlet and to a trocar, where the surgical tool is inserted through the trocar. Optionally, in some embodiments, a filtration system may be arranged between the first inlet and the fluid conduit for filtering out any toxic particles. Optionally, in some embodiments, a fluid trap system may be coupled to the fluid conduit for collecting any liquids, such as blood being sucked into the fluid conduit, thus preventing it from entering the smoke evacuation module.

A plug of the surgical tool may be connected to a first socket at a socket interface of the control system for providing power to the surgical tool. The first valve element may be controlled by the controller to provide a first suction at a flow rate suitable for closed surgery, in particular laparoscopy.

According to an embodiment, the pump system further comprises a second valve element connected to the pump and further to a second inlet at the inlet interface, wherein the controller is configured to control the second valve element to provide a second suction adapted to open surgery.

A second suction mode may be adapted for invasive surgical procedures, such as cardiac or aortic surgery. The term “invasive” should here be understood as any open surgical procedure performed with tools inserted through relatively large incisions in the patient.

A flexible fluid conduit, e.g., a suction tube, may be connected to a second inlet and to the surgical tool, e.g., a diathermy device. Optionally, in some embodiments a filtration system may be arranged at the patient end or distal end of the fluid conduit for filtering out any toxic particles.

A plug of the surgical tool may be connected to a second socket at the socket interface for providing power to the surgical tool. The second valve element may be controlled by the controller to provide a second suction at a flow rate suitable for open surgery.

Alternatively, the surgical tool may be omitted, and the suction tube may simply be connected to the second inlet. Optionally, in some embodiments, a flow regulator or flow restrictor may be arranged along the suction tube for adjusting or reducing the flow rate.

According to an embodiment, the controller is configured to continue at least one of the first and second suctions for at least a first or second lag time after the respective surgical tool is deactivated.

This lag time may ensure that the surgical smoke is removed from the operation site. The lag time may be set by the technician and may be adapted to conditions relating to the surgical procedure. In the first suction mode, the first suction may be continued for a first lag time after the respective surgical tool is deactivated, e.g., by turning it off or not activating the buttons. Similarly, in the second suction mode, the second suction may be continued for a second lag time after the respective surgical tool is deactivated, e.g., by turning it off or not activating the buttons. The lag times may be inputted to the controller using the dedicated user interface.

The first and second valve elements may be electromagnetic operated valves. Other valve elements may also be used.

According to an embodiment, the controller is configured to adjust at least the first or second suction within at least a first or second suction level and/or to adjust at least the first or second lag time within at least a first or second lag time period.

A surgent or technician may set the first suction and/or the second suction using the dedicated user interface. The second suction may be set to a desired flow rate between 60-80 litres per minutes, thus allowing for an optimal flow rate when operating together with the surgical tool. The second suction may also be set to provide a flow rate between 100-150 litres per minutes without a surgical tool connected to the smoke evacuation module.

The first suction may be adjusted to a flow rate between 0-10 litres per minutes, thus allowing for an optimal flow rate. During closed surgery, an insufflator gas, e.g., carbon dioxide, is feed form an insufflator device to the patient. If the insufflator gas is not heated, then the flow rate of the smoke evacuation module should be adjusted to prevent cooling of the patient while maintain the pressure inside the patient. Tests have shown that the flow rate of the first suction may be 5-7 litres per minutes for achieving an optimal balance.

If the insufflator gas is heated, then a higher flow rate of the smoke evacuation module may be selected.

Other tests have shown that the flow rate of the second suction may be about 75 litres per minutes when using an electrosurgical cutter. The flow rate of the second suction may be greater, or smaller, when using a different electrosurgical tool.

According to an embodiment, the pump system is arranged in a first sub-module and the control system is arranged in a separate second sub-module, wherein the first and second sub-modules are configured to be installed inside the supply column and comprise coupling elements configured to engage matching coupling elements in the supply column.

This plug-and-play configuration allows for easy installation in any supply columns from different manufactures. Coupling elements on the first and second sub-modules allows for quick connection of electrical power, compressed gas (e.g., air), etc. The outlet interface may comprise an exhaust port configured to be coupled to an external exhaust system, for example, located in the supply column in some embodiments. The first sub-module may simply be interconnected to the second sub-module via suitable couplings, e.g., electrical terminals.

According to an embodiment, the control system comprises at least one first measuring unit configured to measure a change in a standby parameter of the at least one surgical tool, wherein the change in the standby parameter is caused by an activation of that surgical tool.

Embodiments of the present invention allows for an automatic activation of the smoke evacuation module. A first measuring unit may be arranged relative to each socket or power cable extending to the surgical tool and connected to the controller. The first measuring unit may be configured to measure a change in the standby current, voltage or power. Turning the surgical tool on and/or activating buttons on the surgical tool causes an increase in power demands, which may be detected in the controller as a change in the standby current, voltage or power.

The measured standby parameter is used as a reference value for detecting the change in the standby parameter. Upon detecting a change in the standby parameter, the suction may automatically be turned on by the controller. No need for a separate foot pedal or a current clamp to activate the smoke evacuation module,

According to an embodiment, the control system or pump system comprises at least one second measuring unit configured to measure at least one pressure of the inlet interface, where the smoke evacuation module is configured to calibrate a flow alarm according to the measured pressure.

The present smoke evacuation module may be fitted with a flow alarm for alerting the nurses or surgent that the suction is abnormal. A second measuring unit may be arranged relative to each of the inlets of the inlet interface and may be connected to the controller. The controller may monitor the pressure or flow rate of the first inlet and/or the second inlet using the second measuring unit.

The controller may be configured to perform another calibration procedure, wherein the controller may be calibrated to measure a flow rate or pressure of the surgical tool. The measured flow rate or pressure may then be stored in the controller and used as a reference valve for detecting a drop in the flow rate or pressure. A drop may indicate time for replacement of the filtration system, a blockage of the fluid conduit, etc. The controller may be configured to compare the drop to an alarm threshold, which may be a predetermined percentage of the measured reference value. When the drop exceeds the alarm threshold, then an alarm signal may be transmitted to the flow alarm which may generate a visual alarm and/or an audio alarm.

An aspect of embodiments of the invention is also achieved by a method, comprising:
connecting at least one surgical tool to at least one socket at a socket interface of the control system;
initiating a calibration procedure in the controller by inputting a calibration signal to the controller via a dedicated user interface;
wherein the controller, during calibration, determines a first standby parameter of the at least one surgical tool.

This provides a quick and simple method of calibrating the smoke evacuation module to a selected surgical tool. The smoke evacuation module may be calibrated to ensure that it functions correctly with the selected surgical tool. Before calibration, a first surgical tool is connected to a first socket of the smoke evacuation module. The calibration may then be initiated by inputting a predetermined calibration signal using the user interface.

During calibration, the controller may use a first measuring unit to measure a first reference value, e.g., a standby parameter of a first surgical tool. The measured standby parameter may be zero or a value greater than zero. The first reference value may be stored in the controller and may be used to detect a change in the standby parameter, which may be used to automatically start the first suction. The smoke evacuation module is now ready to function with the first surgical tool.

No exchange of data or handshakes is needed to calibrate the surgical tool, this eliminates the need for establishing a data link between the surgical tool and the smoke evacuation module. Other calibration procedures may also be used to calibrate the smoke evacuation module to function correctly with the surgical tool.

If a second surgical tool is connected to the first socket or a second socket at the socket interface, then the calibration procedure may be repeated for that surgical tool.

During calibration, the controller may use a second measuring unit to measure a second reference value, e.g., a standby parameter of the second surgical tool. The measured standby parameter may be zero or a value greater than zero. The second reference value may be stored in the controller and may be used to detect a change in the standby parameter, which may be used to automatically start the second suction. The smoke evacuation module is now ready to function with the second surgical tool.

Similarly, the calibration procedure is repeated if the first or second surgical tool is removed and replaced with another surgical tool, or later re-connected to the smoke evacuation module.

According to an embodiment, the method further comprises:
further connecting the at least one surgical tool to at least one inlet at the inlet interface;
initiating another calibration procedure in the controller by inputting another calibration signal to the controller via the dedicated user interface;
wherein the controller, during calibration, determines a flow rate or pressure of the at least one surgical tool, which is used in the controller to detect a drop in the flow rate or pressure exceeding an alarm threshold.

Embodiments of the present invention also allow the flow alarm of the smoke evacuation module to be calibrated to the suction level of the surgical tool. Also, the flow alarm can be quickly recalibrated to match the suction level of another surgical tool.

Before calibration, the first or second surgical tool is further connected to the first or second inlet at the inlet interface. The calibration is initiated by inputting another calibration signal using the dedicated user interface. During calibration, the controller uses another measuring unit to measure a flow rate or pressure of that surgical tool. The measured flow rate or pressure is then stored in the controller and used as a reference value for detecting a drop in the flow rate or pressure of that surgical tool. The flow alarm of the smoke evacuation module is now calibrated and ready to function correctly with that surgical tool.

During the first or second suction mode, the measured drop in the flow rate or pressure is compared with an alarm threshold stored in the controller. The alarm threshold may be defined as a predetermined percentage of the reference value. The alarm threshold and other parameters in the controller may be adjusted via a programming interface on the control system. This alerts the nurses or surgeons to any abnormal suction in the smoke evacuation module.

An aspect of embodiments of the invention is further achieved by a supply column, comprising:
at least an air source, main power lines, or an exhaust system;
a smoke evacuation module installed inside the supply column, wherein the smoke evacuation module comprises a pump system and a control system, the pump system or the control system being coupled to the air source, the main power lines or the exhaust system.

This provides a compact solution where the smoke evacuation module is arranged inside the supply column and coupled to the supply column. In embodiments, the smoke evacuation module may be installed using a plug and play engagement between the smoke evacuation module and the supply column, as described above. This provides a versatile solution which can be installed in supply columns from different manufactures and/or is able to operate with surgical tools of different manufactures.

### BRIEF DESCRIPTION

Some of the embodiments will be described in detail, with references to the following Figures, wherein like designations denote like members, wherein:

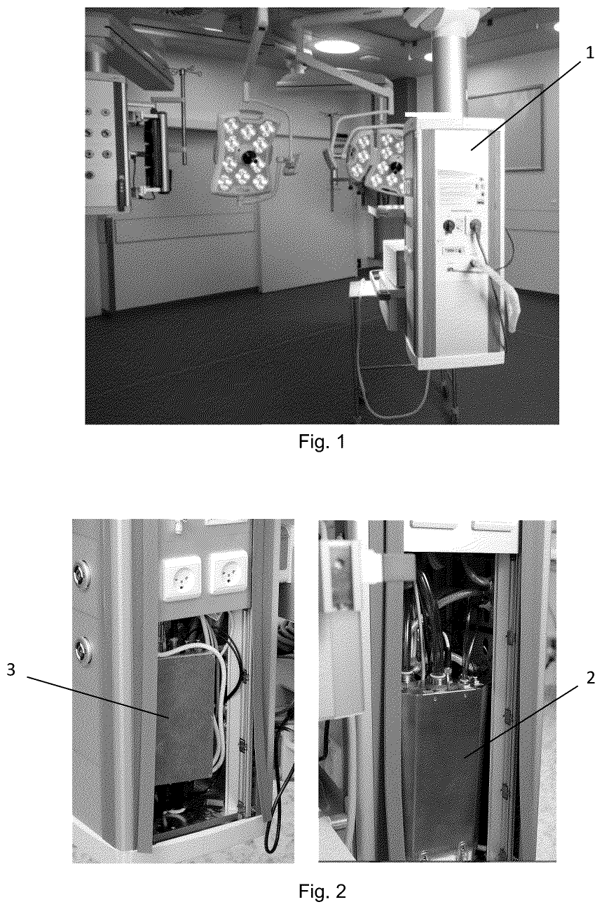

FIG. 1 shows an exemplary embodiment of a supply column in an operating room;

FIG. 2 shows a smoke evacuation module adapted to plug-and-play engagement with the supply column shown in FIG. 1;

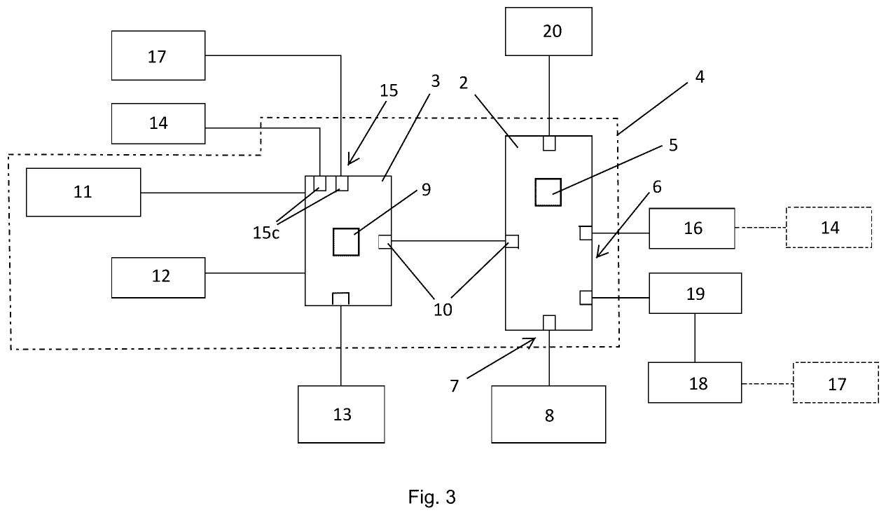

FIG. 3 shows a block diagram of the smoke evacuation module installed in a supply column and connected to two surgical tools;

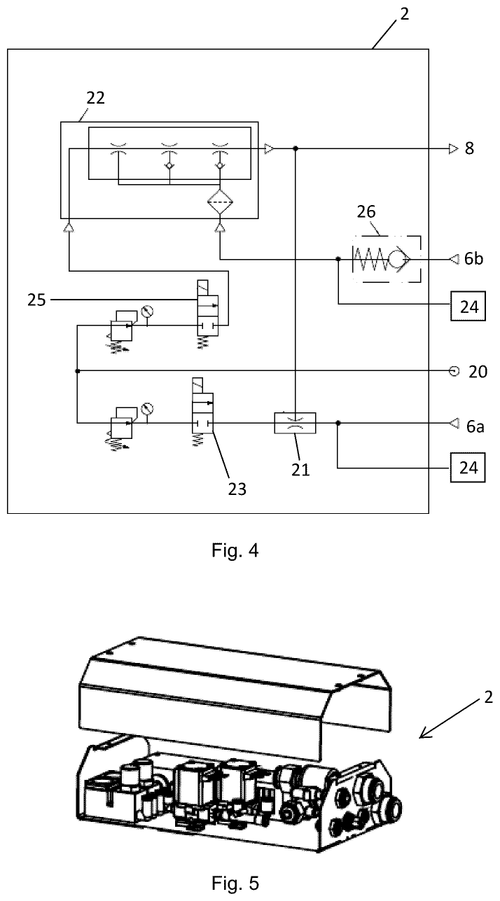

FIG. 4 shows a block diagram of the pump system;

FIG. 5 shows the pump system arranged in a sub-module;

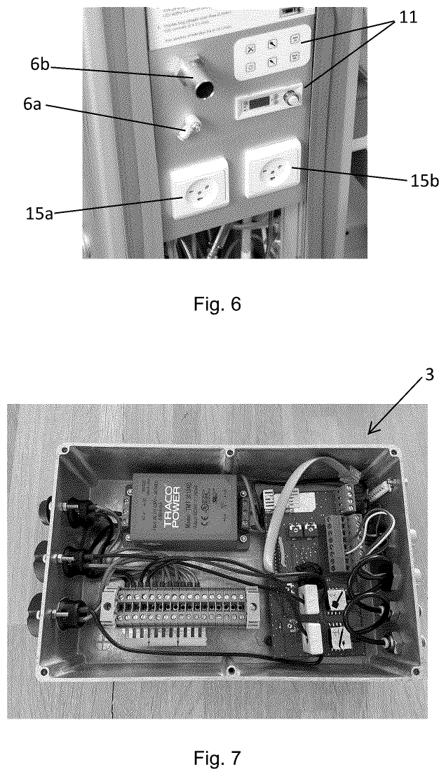

FIG. 6 shows an exemplary embodiment of the user interface, the inlet interface and the socket interface;

FIG. 7 shows the control system arranged in a sub-module;

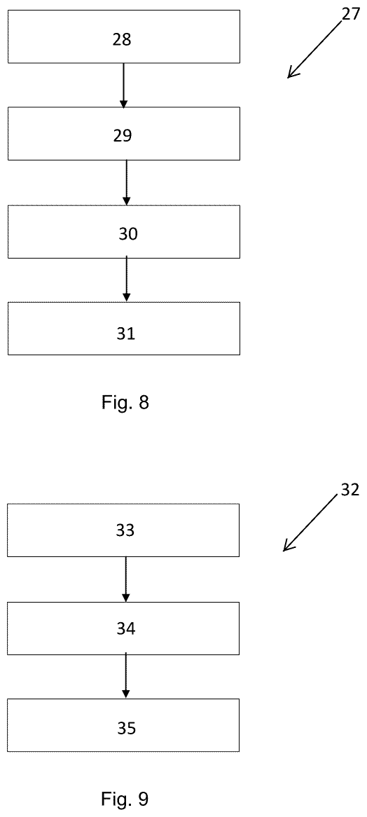

FIG. 8 shows a flow chart of a calibration procedure for calibrating the smoke evacuation module; and

FIG. 9 shows a flow chart of a calibration procedure for calibrating a flow alarm of the evacuation module.

### REFERENCE LIST

1 Supply column
2 Pump system
3 Control system
4 Smoke evacuation module
5 Pump
6 Inlet interface
6a First inlet
6b Second inlet
7 Outlet interface
8 Exhaust
9 Controller
10 Coupling elements
11 User interface
12 Flow meters
13 Main power lines
14 First surgical tool
15 Socket interface
15a First socket
15b Second socket
15c First measuring unit
16 Filtration system
17 Second surgical tool
18 Filtration system
19 Fluid trap
20 Air source
21 First vacuum ejector
22 Second vacuum ejector
23 First valve element
24 Second measuring unit
25 Second valve element
26 Flow regulator
27 Calibration procedure
28 Connecting surgical tool
29 Initiate calibration procedure
30 Calibrate smoke evacuation module
31 Continue to next calibration procedure
32 Calibration procedure
33 Initiate calibration procedure
34 Calibrate flow alarm
35 End of calibration procedure

### DETAILED DESCRIPTION

FIG. 1 shows an exemplary embodiment of a supply column 1 in an operating room, in which a smoke evacuation module according to embodiments of the present invention can be installed. The supply column 1 is coupled to an air source (not shown), mains (not shown) and an exhaust system (not shown).

FIG. 2 shows a smoke evacuation module adapted to plug-and-play engagement with the supply column shown in FIG. 1, wherein the smoke evacuation module comprises a pump system 2 and a control system 3 configured to be coupled to the pump system 2.

FIG. 3 shows a block diagram of the smoke evacuation module 4 installed in the supply column 1 and two surgical tools connected to the smoke evacuation module 4.

The pump system 2 comprises a pump 5 configured to generate a suction flow from an inlet interface 6 to an outlet interface 7. The inlet interface 6 is configured to operatively engage with one or more surgical tools. The outlet interface 7 is configured to engage the exhaust 8.

The control system 3 comprises a controller 9 configured to operate the pump system 2 in at least two different suction modes. The control system 3 is configured to be connected to the pump system 2 via matching coupling elements 10. The control system 3 is connected to a dedicated user interface 11 on which a user is able to input signals to the controller 9. The control system 3 is further connected to flow meters 12 adapted to indicate the flow rate in the smoke evacuation module 4.

The control system 3 is configured to be connected to main power lines 13 via matching coupling elements.

A first surgical tool 14 is configured to be connected to a socket interface 15 of the control system 3. The first surgical tool 14 is further configured to be coupled to the inlet interface 6 via a first fluid conduit. A filtration system 16 is arranged between the inlet interface 6 and the first surgical tool 14.

A second surgical tool 17 is also configured to be connected to the socket interface 15. The second surgical tool 17 is further configured to be coupled to the inlet interface 6 via a second fluid conduit. A filtration system 18 and/or a fluid trap 19 is arranged between the inlet interface 6 and the second surgical tool 17.

The pump system 2 is configured to be coupled to the air source 20 via matching coupling elements. The air source 20 is configured to supply a compressed gas, e.g., air, to the pump system 2.

FIG. 4 shows a block diagram of the pump system 2. The pump 5 comprises a first vacuum ejector 21 and a second vacuum ejector 22. The first vacuum ejector 21 is connected to a first valve element 23 at one end and to a first inlet 6a at the other end. The first vacuum ejector 21 is configured to generate a first suction at a first inlet 6a. A second measuring unit 24 is arranged at the first inlet 6a for measuring the flow rate or pressure of the first suction. The second vacuum ejector 22 is connected to a second valve element 25 at one end and to a flow regulator 26 at the other end. The second vacuum ejector 22 is configured to generate a second suction at a second inlet 6b. A second measuring unit 24 is arranged at the second inlet 6b for measuring the flow rate or pressure of the second suction. The first and second suction is controlled by the controller 9.

FIG. 5 shows the pump system 2 arranged in a sub-module adapted to be installed in the supply column 1. The pump system 2 is arranged in a housing where the various coupling elements are located at the housing wall for easy coupling to the air source 20, exhaust 8 and to the control system 3.

FIG. 6 shows an exemplary embodiment of the user interface 11, the inlet interface 6 and the socket interface 15. Here the inlet interface 6 comprises a first inlet 6a and a second 6b adapted to be connected to the first and second surgical tools 14, 17 respectively.

Here the socket interface 15 comprises a first socket 15a and a second socket 15b adapted to be connected to the first and second surgical tools 14, 17 respectively.

FIG. 7 shows the control system 3 arranged in a sub-module adapted to be installed in the supply column 1. The control system 3 is arranged in a housing where the various coupling elements are located at the housing wall for easy coupling to the main power lines 13, the flow metres 12, the user interface 11 and to the pump system 2.

FIG. 8 shows a flow chart of a calibration procedure 27 for calibrating the smoke evacuation module 4. Before calibration, the first surgical tool 14 is connected 28 to the first socket 15a and to the first inlet 6a.

The calibration procedure is then initiated 29 by inputting a first calibration signal on the user interface 11. The controller 9 performs the calibration 30 by measuring a standby parameter of the first surgical tool 14 using a first measuring unit 15c. The standby parameter is stored and used as a reference value to detect a change in the standby parameter during operation.

Once the controller 9 has completed the calibration, the user can continue 31 to calibrate a flow alarm of the smoke evacuation module 4. The flow alarm generates an alarm when the flow rate or pressure drops below an alarm threshold.

FIG. 9 shows a flow chart of a calibration procedure 32 for calibrating the flow alarm of the evacuation module 4. The calibration procedure is initiated 33 by inputting a second calibration signal on the user interface 11. The controller 9 perform the calibration 34 by measuring a flow rate or pressure of the first surgical tool 14 using the second measuring unit 24. The flow rate or pressure is stored and used as a reference value to detect a drop in the flow rate or pressure during operation.

Once the controller 9 has completed the calibration, the smoke evacuation module 4 is ready to use 35 and will function correctly with the first surgical tool 14. The calibration procedures 27, 32 is repeated for the second surgical tool 17.

Although the present invention has been disclosed in the form of embodiments and variations thereon, it will be understood that numerous additional modifications and variations could be made thereto without departing from the scope of the invention.

For the sake of clarity, it is to be understood that the use of “a” or “an” throughout this application does not exclude a plurality, and “comprising” does not exclude other steps or elements. The mention of a “unit” or a “module” does not preclude the use of more than one unit or module.

1. A smoke evacuation module configured for plug-and-play engagement with a supply column; the smoke evacuation module comprising:
a pump system connected to an inlet interface at one end and to an outlet interface at the other end, the pump system comprising a pump configured to generate a suction flow from the inlet interface to the outlet interface,
wherein the inlet interface is configured to operatively engage with at least one surgical tool;
wherein the outlet interface is configured to engage with an exhaust; and
a control system configured to be connected to the pump system, the control system comprising a controller configured to operate the pump system in at least different two suction modes.

2. The smoke evacuation module according to claim 1, wherein the smoke evacuation module is configured to be installed inside the supply column, wherein at least the pump system or the control system is configured to be coupled to the supply column.

3. The smoke evacuation module according to claim 1, wherein the pump is at least one vacuum ejector.

4. The smoke evacuation module according to claim 3, wherein the pump is powered by an external air source configured to supply compressed air to the pump.

5. The smoke evacuation module according to claim 1, wherein the controller is configured to perform a calibration procedure, wherein the smoke evacuation module is calibrated to function correctly with the at least one surgical tool in either of the at least two different suction modes.

6. The smoke evacuation module according to claim 1, wherein the inlet interface comprises at least two inlets each configured to be coupled a dedicated surgical tool.

7. The smoke evacuation module according to claim 5, wherein the pump is configured to operatively engage at least two surgical tools coupled to the inlet interface at the same time, wherein the controller is configured to independently control respective suctions provided to the at least two surgical tools.

8. The smoke evacuation module according to claim 8, wherein the pump system comprises a first valve element connected to the pump and further to a first inlet at the inlet interface, wherein the controller is configured to control the first valve element to provide a first suction configured to closed surgery.

9. The smoke evacuation module according to claim 8, wherein the pump system further comprises a second valve element connected to the pump and further to a second inlet at the inlet interface, wherein the controller is configured to control the second valve element to provide a second suction configured to open surgery.

10. The smoke evacuation module according to claim 9, wherein the controller is configured to continue at least one of the first and second suctions for at least a first or second lag time after the respective surgical tool is deactivated.

11. The smoke evacuation module according to claim 10, wherein the controller is configured to adjust at least the first or second suction within at least a first or second suction level and/or to adjust at least the first or second lag time within at least a first or second lag time period.

12. The smoke evacuation module according to claim 1, wherein the pump system is arranged in a first sub-module and the control system is arranged in a separate second sub-module, wherein the first and second sub-modules are configured to be installed inside the supply column and comprise coupling elements configured to engage matching coupling elements in the supply column.

13. The smoke evacuation module according to claim 1, wherein the control system comprises at least one first measuring unit configured to measure a change in a standby parameter of the at least one surgical tool, wherein the change in the standby parameter is caused by an activation of that surgical tool.

14. The smoke evacuation module according to claim 1, wherein the control system or pump system comprises at least one second measuring unit configured to measure at least one pressure of the inlet interface, wherein the smoke evacuation module is configured to calibrate a flow alarm according to the measured pressure.

15. A method of calibrating the smoke evacuation module according to claim 1, comprising:
connecting the at least one surgical tool to at least one socket at a socket interface of the control system;
initiating a calibration procedure in the controller by inputting a calibration signal to the controller via a dedicated user interface; and
wherein the controller, during calibration, determines a first standby parameter of the at least one surgical tool.

16. The method of calibrating the smoke evacuation module according to claim 15, wherein the method further comprises:
further connecting the at least one surgical tool to at least one inlet at the inlet interface;
initiating another calibration procedure in the controller by inputting another calibration signal to the controller via the dedicated user interface; and
wherein the controller, during calibration, determines a flow rate or pressure of the at least one surgical tool, which is used in the controller to detect a drop in the flow rate or pressure exceeding an alarm threshold.

17. A supply column comprising:
at least an air source, main power lines, or an exhaust system;
a smoke evacuation module installed inside the supply column, wherein the smoke evacuation module comprises a pump system and a control system, the pump system or the control system being coupled to the air source, the main power lines or the exhaust system.
